(19) United States
(12) Patent Application Publication
(10) Pub. No.: US 2018/0043585 A9
(43) Pub. Date: Feb. 15, 2018

(54) DELIVERY OF PARTICLES USING HYGROSCOPIC EXCIPIENTS

(71) Applicants: VIRGINIA COMMONWEALTH UNIVERSITY, Richmond, VA (US); ARGEAUX INC., San Diego, CA (US)

(72) Inventors: Philip Worth LONGEST, Midlothian, VA (US); Michael HINDLE, North Chesterfield, VA (US); Elaine M. PHILLIPS, San Diego, CA (US)

(21) Appl. No.: 15/022,589

(22) PCT Filed: Sep. 19, 2014
(86) PCT No.: PCT/US14/56477

§ 371 Date: Mar. 17, 2016

Related U.S. Application Data
(60) Provisional application No. 61/880,613, filed on Sep. 20, 2013.

(51) Int. Cl. B29C 43/00; B29C 35/02; A61L 27/50; A61F 2/24; A61L 27/36

(52) U.S. Cl. CPC B29C 43/003; A61F 2/2412; A61F 2/2415; A61L 27/3691; A61L 27/3687; A61L 27/367; A61L 27/50; B29C 35/0261; A61L 2430/20; A61L 2430/40; A61L 2400/18; B29L 2031/753

(57) ABSTRACT

A method of delivering a therapeutic agent comprises providing an aerosol generator that produces an aerosol of particles or droplets containing a therapeutic agent and an excipient for delivery to a first portion of a respiratory tract. The particles or droplets have an initial diameter from about 1 μm to about 8 μm, and the initial momentum of the particles or droplets minimizes deposition in the first portion. The particles or droplets are exposed to relative humidity in the respiratory tract by delivering them at a flow rate that defines the residence time in the respiratory tract. The particles or droplets increase in diameter due to hygroscopic growth caused by relative humidity. The aerosol is nasally exhaled so that particles or droplets are delivered to the nasal cavity and deposited in the nasal turbinates or sinus in part because of their increased diameter.

### CROSS-REFERENCE

This application claims priority to U.S. Provisional Application 61/880,613 filed Sep. 20,2013.

### BACKGROUND OF THE INVENTION

### 1. Field of the Invention

The present invention generally relates to medical devices and methods, and more particularly relates to devices and methods for improved delivery of particles or droplets to a patient along the patient's respiratory tract. The particles or droplets preferably contain a therapeutic agent and a hygroscopic excipient.

Inhaled delivery of particles or droplets provides an advantageous route for administration of therapeutic agents locally as well as systemically. A number of medical conditions may be treated by direct application of a therapeutic agent to targeted sites in the nasal cavity. For example, local treatment of rhinitis, sinusitis, congestion and nasal polyps may be accomplished by delivering therapeutic agents to portions of the nasal cavity.

The nose may also be used to deliver the therapeutic agents to another part of the body such as the brain. This may provide more direct delivery of the therapeutic agent to the systemic circulation and in some cases avoids first pass metabolism by the liver. Additionally, the nasal cavity can be a route for rapid uptake of the therapeutic agent into the systemic circulation. Other conditions which may be treated using nasal delivery of drugs include pain, infection, seizures, anxiety, emesis, cognitive diseases such as Alzheimer's, as well as other diseases such as Parkinson's disease, hepatitis, growth failure, obesity, and a host of other conditions.

However, delivering therapeutic agents nasally is currently not an effective delivery method. It is challenging to deliver the therapeutic agent accurately and with the correct dosage to a desireted target region. It would therefore be desirable to provide improved devices and methods that allow delivery of therapeutic agents more effectively along a respiratory tract such as in the nasal cavity. At least some of these objectives will be satisfied by the devices and methods disclosed below.

### 2. Description of the Background Art

Journal articles related to delivery of particles include “Condensational growth of combination drug-excipient submicrometer particles for targeted high-efficiency pulmonary delivery: evaluation of formulation and delivery device,” by Michael Hindle and P. Worth Longest, Journal of Pharmacy and Pharmacology, Vol. 64, Issue 9, pp. 1254-1263, September 2012; and “Current understanding of nasal morphology and physiology as a drug delivery target,” by Julie D. Suman, Drug Deliv. Transl. Res. (2013) 3:4-15. Patents and patent publications related to delivery of particles include U.S. Patent Publication Nos. 2012/0251594; and 2013/0008437; and U.S. Pat. No. 8,479,728; the entire contents of which are incorporated herein by reference.

### SUMMARY OF THE INVENTION

The present invention generally relates to medical devices and methods, and more particularly relates to delivery of particles or droplets to a patient, and even more particularly relates to delivery of particles or droplets using a hygroscopic excipient.

In a first aspect of the present invention, a method of delivering a therapeutic agent to targeted regions of a nasal cavity in a patient comprises providing a generator for generating an aerosol comprising particles or droplets containing a therapeutic agent and an excipient, and delivering the aerosol to a first portion of a respiratory tract in the patient. The combination particles or droplets have an initial diameter from about 1 μm to about 8 μm. The initial momentum of the particles or droplets minimizes deposition of the particles or droplets in the first portion of the respiratory tract. The first portion of the respiratory tract may be the mouth or the nasal cavity. The method also comprises exposing the particles or droplets to relative humidity in the respiratory tract by delivering the particles or droplets at a flow rate with an optional breath hold that defines the residence time of the particles or droplets in the respiratory tract, and increasing diameter of the particles or droplets due to hygroscopic growth caused by the relative humidity and residence time in the respiratory tract. The method also comprises nasally exhaling a volume of the aerosol sufficient to deliver particles or droplets to the nasal cavity and minimize pulmonary delivery thereof, and depositing the increased diameter particles or droplets in the nasal turbinates and/or the sinus cavities of the nasal cavity enhanced by the increased diameter of the particles or droplets.

The first portion of the respiratory tract may be a mouth, an oropharyngeal region, a trachea, a pharynx, or a nasal cavity of the patient.

The particles or droplets may further comprise a hygroscopic excipient such as a salt, a sugar, an acid, a buffer, a glycol, or a lactam. Exemplary hygroscopic excipients may comprise one or more of the following materials: sodium chloride, sodium citrate, citric acid, potassium chloride, zinc chloride, calcium chloride, magnesium chloride, potassium carbonate, potassium phosphate, carnallite, ferric ammonium citrate, magnesium sulfate, sodium sulfite, calcium oxide, ammonium sulfate, sorbital, mannitol, glucose, maltose, galactose, fructose, sucrose, polyethylene glycol, propylene glycol, glycerol, sulfuric acid, malonic acid, adipic acid; lactams such as 2-pyrrolidone, polyvinylpolyprrolidone (PVP), potassium hydroxide, sodium hydroxide, gelatin, hydroxypropyl methylcellulose, pullalan, starch, polyvinyl alcohol, and sodium cromoglycate.

The therapeutic agent may or may not promote hygroscopic growth of the particles or droplets, and the initial momentum of the particles or droplets may permit substantially unimpeded travel of the particles or droplets through the first portion of the respiratory tract. The therapeutic agent may have an initial particle size in the range of 10 nm to 8 μm which is formulated within a combination particle or droplet containing one or more hygroscopic excipient or other excipients that are recognized by those skilled in the art as necessary to form stable droplets or particles such as surfactants, dispersion enhancers, bulking agents, lubricants. The increased diameter of the particles or droplets may substantially enhance deposition of the particles or droplets in the nasal turbinates and/or sinus cavities.

The therapeutic agent may be selected from the group consisting of agents for the treatment of asthma, rhinitis, chronic sinusitis and other respiratory disorders, anesthesia agents, nucleic acid molecules, anti-pain agents, anti-inflammation agents, anti-depressants and other mood altering drugs, anti-viral agents, anti-bacterial agents, anti-fungal agents, anti-cancer agents, hormones, benzodiazepines and calcitonin.

Relative humidity may be the natural relative humidity in a portion of the respiratory tract. The ratio of the increased diameter to the initial diameter may range from about 2 to about 20. The initial momentum may be consistent with the inhalation of the 1-8 μm particles or droplets at a flow rate of less than about 30 liters per minute. In other embodiments the flow rate may be less than about 20 liters per minute, or it may be less than about 10 liters per minutes. The use of the term “particles” and “droplets” may be interchanged with one another in this specification.

In another aspect of the present invention, a device for delivering aerosolized particles or droplets to a region of a nasal passageway in a patient comprises an aerosol generator, an apparatus to control a volume of aerosol introduced to a respiratory tract from the aerosol generator, a therapeutic agent and a hygroscopic excipient. The therapeutic agent and the excipient are contained in the aerosol generator, and the aerosol generator generates an aerosol of particles or droplets containing the therapeutic agent and the hygroscopic excipient. The particles or droplets have an initial momentum while increasing in diameter to permit substantially unimpeded travel of the particles or the droplets through a first portion of the respiratory tract while minimizing aerosol deposition. Exposure of the particles or droplets to relative humidity in the respiratory tract increases diameter of the particles or droplets to a size that generally favors deposition in the nasal passageway. The increased diameter of the particles or droplets results in deposition of the particles or droplets in the nasal turbinates and/or the sinus cavities of the nasal passageway.

The aerosol generator may be any one of a number of different generators including but not limited to a metered dose inhaler, a dry powder inhaler, a liquid spray device, a capillary aerosol generator, a condensational aerosol generator, a jet nebulizer, or an ultrasonic nebulizer. The aerosol generator may provide a single bolus of aerosol or a series of intermittent boluses of aerosol. In some embodiments, a continuous aerosol may be provided by the generator, or the continuous aerosol generator may be operated in an intermittent mode.

The apparatus that controls the volume of aerosol may also control the rate of aerosol delivery to the respiratory tract, and it may also inject a volume of gas at positive pressure. The apparatus that controls the volume of aerosol may also limit the rate and volume of air inhaled by the patient, and this may be accomplished with a chamber containing ambient or conditioned air or another gas. The chamber preferably is positioned behind the aerosol generator. In other embodiments, a spacer may be used to control the volume of aerosol, and the spacer is preferably positioned in front of the aerosol generator. The air inhaled by the patient may be limited to from about 25 mL to about 250 mL.

The therapeutic agent may be selected from the group consisting of agents for the treatment of asthma and other respiratory disorders, anesthesia agents, nucleic acid molecules, anti-pain agents, anti-inflammation agents, anti-depressants and other mood altering drugs, anti-viral agents, anti-bacterial agents, anti-fungal agents, anti-cancer agents, hormones, benzodiazepines and calcitonin. The therapeutic agent may or may not promote hygroscopic growth of the particles or droplets.

The hygroscopic excipient may comprise a salt, a sugar, an acid, a buffer, a glycol, or a lactam. Exemplary embodiments of the hygroscopic excipient may comprise one or more of the following materials: sodium chloride, sodium citrate, citric acid, potassium chloride, zinc chloride, calcium chloride, magnesium chloride, potassium carbonate, potassium phosphate, carnallite, ferric ammonium citrate, magnesium sulfate, sodium sulfite, calcium oxide, ammonium sulfate, sorbital, mannitol, glucose, maltose, galactose, fructose, sucrose, polyethylene glycol, propylene glycol, glycerol, sulfuric acid, malonic acid, adipic acid; lactams such as 2-pyrrolidone, polyvinylpolyprrolidone (PVP), potassium hydroxide, sodium hydroxide, gelatin, hydroxypropyl methylcellulose, pullalan, starch, polyvinyl alcohol, and sodium cromoglycate.

The ratio of the increased diameter to the initial diameter of the particles or droplets may range from about 2 to about 20. The device may deliver a fixed volume of the aerosol to first portion of the respiratory tract. This volume should be sufficient to deliver the particles or droplets to the nasal cavity while at the same time being low enough so as not to deliver the aerosol to the deep lung. The fixed volume may range from about 25 mL to about 250 mL. The device may control inhalation flow rate of the aerosol delivered to the respiratory tract and the flow rate may range from about 1 liter per minute to about 30 liters per minute. The flow rate may minimize pulmonary deposition and may maximize deposition of the particles or droplets in the nasal cavity. The particles or droplets may also be deposited in a nasal ostium, a nasopharynx, an olfactory region, or in an area posterior relative to the vestibule.

In still another aspect of the present invention, use of a therapeutic agent and an excipient for treating diseases comprises a therapeutic agent and an excipient delivered from a generator to form particles or droplets delivered in an aerosol to targeted nasal tissue along a nasal passageway in a patient. The particles or droplets have an initial momentum to minimize deposition of the particles or droplets in a first region of a respiratory tract away from the targeted nasal tissue, and the particles or droplets increase in diameter when exposed to relative humidity in a portion of the respiratory tract. The increased diameter of the particles or droplets enhances deposition of the particles in the targeted nasal tissue, and the targeted nasal tissue comprises the nasal turbinates and/or sinus cavities of the nasal passageway.

The therapeutic agent may be selected from the group consisting of agents for the treatment of asthma and other respiratory disorders, anesthesia agents, nucleic acid molecules, anti-pain agents, anti-inflammation agents, anti-depressants and other mood altering drugs, anti-viral agents, anti-bacterial agents, anti-fungal agents, anti-cancer agents, hormones, benzodiazepines and calcitonin. The ratio of the increase diameter to the initial diameter of the particles or droplets may range from about 2 to about 20.

In yet another aspect of the present invention, a method of providing one or more agents to a posterior region of a subject s nose comprises the steps of generating an aerosol and delivering the aerosol to the subject's respiratory tract. The aerosol comprises a plurality of particles or droplets containing the one or more agents and optionally one or more excipients. The particles or droplets have a diameter ranging from 1 μm to 8 μm upon generation, the particle size of the therapeutic agent ranging from 10 nm to 8 μm, and also have a property of hygroscopic growth when exposed to a humid environment. The aerosol is delivered to the respiratory tract at a predetermined air flow rate, and delivery is performed for a period sufficient to at least partially fill one or more of the subject s nasal cavity, pharynx, larynx, and trachea. The particles or droplets experience hygroscopic growth due to exposure to relative humidity in one or more of the subject s nasal cavity, pharynx, larynx, and trachea. After the period, the subject exhales through the nose so that a majority of the particles or droplets deposit in the posterior region of the subject's nose.

In some embodiments, the predetermined inhalation air flow rate may range from about 1 to about 30 liters per minute.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic diagram of basic nasal anatomy.

FIG. 2 illustrates further details of the nasal anatomy.

FIGS. 3A-3B illustrate cross-sections of the nasal cavity illustrated in FIG. 2.

FIGS. 4A-4F illustrate computational fluid dynamic modeling of particle deposition in one exemplary method.

FIGS. 5A-5F illustrate computational fluid dynamic modeling of particle deposition in another exemplary method.

FIGS. 6A-6E illustrate computational fluid dynamic modeling of particle deposition in yet another exemplary method.

FIGS. 7A-7E illustrate computational fluid dynamic modeling of particle deposition in still another exemplary method.

FIGS. 8A-8B illustrate actuation of an aerosol with chamber.

FIG. 9 illustrates another exemplary embodiment of a chamber.

FIG. 10 illustrates a schematic diagram of an exemplary method of particle delivery.

### DETAILED DESCRIPTION OF THE INVENTION

Specific embodiments of the disclosed device and method will now be described with reference to the drawings. Nothing in this detailed description is intended to imply that any particular component, feature, or step is essential to the invention.

The nose has evolved to condition incoming air (heating and humidifying) as well as to filter particulates in inhaled gases. Thus, the therapeutic agents can be filtered out of the incoming air or are not always delivered to the desired target in the desired dose. Additionally, the therapeutic agents which may be delivered in droplet or particle form are often either too large and therefore deposit too early along the delivery route, or the droplets or particles may be too small and therefore penetrate through the target region or are exhaled instead of depositing in the treatment region. Furthermore, the particles or droplets often are not sized properly to navigate along a tortuous path further creating challenges to accurate delivery of the droplets or particles to the treatment region.

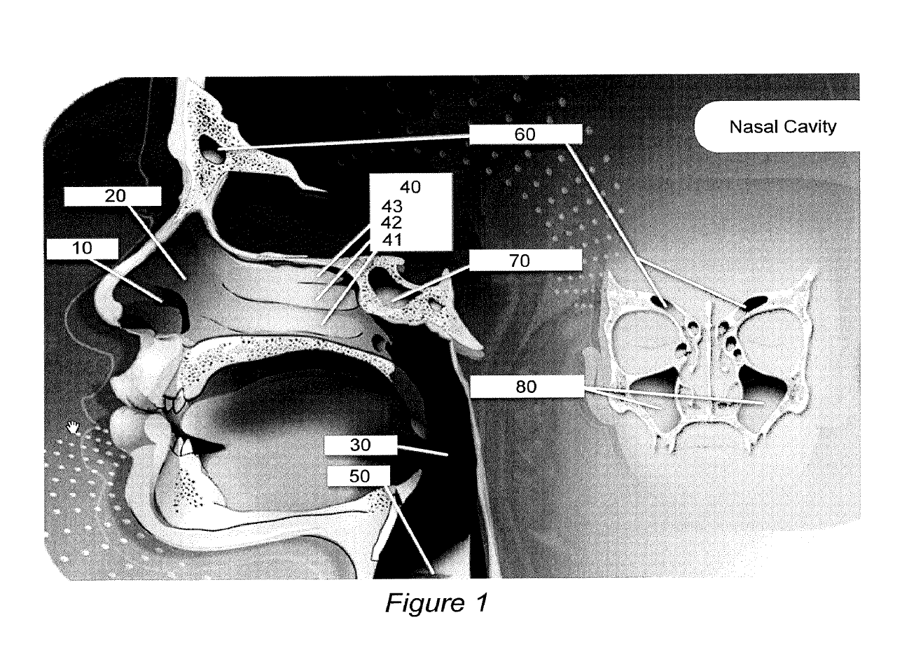

FIG. 1 illustrates a schematic diagram of the human nasal cavity. Two nostrils, also referred to as the nasal vestibule 10 are at the entrance to the nasal cavities 20. At the end of the nasal vestibule 10, the diameter of each cavity decreases at a point known as the nasal ostium. The nasal septum separates the two cavities which extend 12-14 cm from the nostrils to the junction between the nose and pharynx 30. This junction is known as the nasopharynx. The cavities join to form a single passageway at the nasopharynx. Within the nose itself, the main nasal passage is further divided by three projections from the nasal walls called turbinates 40. The three turbinates include the inferior 41, middle 42 and superior 43 turbinate, and they increase the total surface area of the nasal cavity 20 which allows for more efficient humidification of inhaled air. Nasal mucosa lines the nasal cavity 20 and the mucosa is swept toward the back of the nose and drains into the nasopharynx by motion of cilia. The nasal vestibule 10 and leading edge of the turbinates 40 are generally not ciliated. The larynx 50 is located below the pharynx 30. Sinus cavities surround the nasal cavity (sinus frontaux 60, sphenoidal sinus 70, and maxillary sinus 80), and they drain into the nasal cavity 20 by small ostia. The olfactory epithelium which provides a sense of smell is located near the superior turbinate 43 and adjacent the nasal septum.

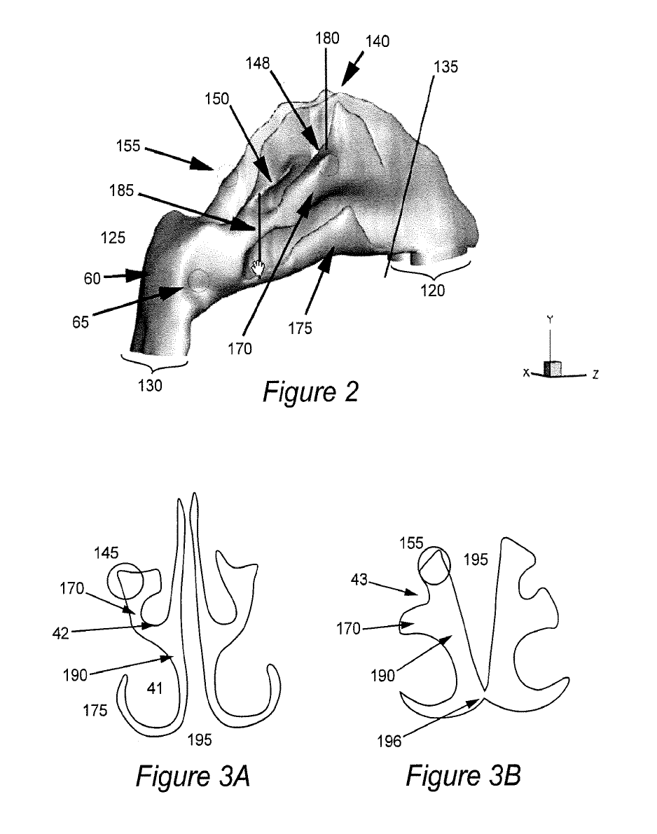

FIG. 2 illustrates the nasal anatomy in greater detail and includes target regions for delivery of therapeutic agents. FIG. 2 includes the nostrils 120, nasopharynx 125, outlet to pharynx 130, and anterior nose (region of vestibule and valve) 135 with arrows pointing to the specific locations of the olfactory region 140, maxillary sinus ostium 145, superior meatus 150, sphenoethmoidal recess 155, adenoids 160, eustachian tube orifice 165, middle meatus (flow passage) 170, and inferior meatus 175. The cross-section lines A-A 180 and B-B 185 are also shown.

FIG. 3A is a cross-section taken along the line A-A 180 in FIG. 2 and highlights the area around the maxillary sinus ostium 145. A majority of flow moves through the middle passage 190, which is divided by the septum 195. The middle turbinate 42 and inferior turbinate 41 extend into the passage and limit flow into the middle meatus 170 and inferior meatus 175. The region of the maxillary sinus ostium 145 is highlighted with a circle.

FIG. 3B is a cross-section taken along the line B-B 185 in FIG. 2 and it highlights the area around the superior turbinate 43. The middle meatus 170 and inferior meatus 175 passages are ending and the middle passage 190 is wider than at cross-section A-A as seen in FIG. 3A. The primary target regions in this view are the superior turbinate 43 and the sphenoethmoidal recess 155. The end of the septum 196 is also indicated with an arrow.

### Deposition of Particles

Without being bound by any theory, it is believed that inhaled particles and droplets deposit in the respiratory tract by three mechanisms including inertial impaction, gravitational sedimentation, and Brownian diffusion. When delivered to the front of the nose, inertial impaction is the most predominant for existing commercial nasal products because the air passageway constricts sharply about 1.5 cm into the nose at the nasal ostium and this results in acceleration of inhaled air and increased turbulence. Additionally, the air stream must change direction at this constriction to enter the turbinate region. Thus, particles that are large or moving at high velocity cannot follow the air stream as it changes direction due to their high momentum and therefore the particles continue along their original direction and inertially impact the airway walls. Similarly, drug laden droplets are often very large and they often inertially impact in the anterior third of the nasal cavity. Particles deposited in the nasal vestibule and anterior regions are physically removed by nasal dripping, blowing or sneezing by the subject/patient. Particles or droplets deposited in the lower posterior region are cleared in the pharynx and may be swallowed. Once the particle deposits in the nasal mucosa, it may exert a local effect or may be absorbed into the blood stream. Thus it is clear that particle momentum is important in controlling particle or droplet deposition along the nasal cavity. Momentum is the product of mass and velocity, and velocity is affected by flow rate and cross-sectional area. The present invention changes momentum of the particles or droplets to control deposition in the respiratory tract. Preferably, momentum is changed by altering size of the particles or droplets.

### Particle Delivery

A patient may inhale the therapeutic agent through the mouth (also referred to as oral inhalation) and from any number of generators that produce an aerosol containing the therapeutic agent and excipients. The aerosol may include drug suspensions, droplets with solubulized therapeutic agent and fine particles from devices such as dry powder inhalers (DPI), metered dose inhalers (MDI) such as the commercially available Respimat device, nebulizers, capillary aerosol generators, etc. The volume and rate of inhaled aerosol may be controlled so that the particles or droplets enter the oropharynx but the volume is generally insufficient to allow entry of the aerosol beyond the trachea-bronchial region and into the pulmonary region. The volumes for airways in an average adult male are known in the art. For a medium adult male, the volume of the mouth-throat region is generally about 60 cc. The volume for an adult male from the trachea to bronchial bifurcation B3 region is about 65 cc, and from the trachea to bronchial bifurcation B6 region is about 95 cc. In embodiments of the present invention, an apparatus controls the volume to restrict delivered volume from the aerosol generator in the range of about 25 cc-250 cc, and in some embodiments this may be 25 cc to 150 cc. Therefore, if the volume of aerosol is limited to about 250 cc or less, the aerosol remains mainly in oropharynx and will not penetrate beyond the trachea-bronchial region.

The patient may inhale slowly via the mouth, pause and then exhale through the nose, or the patient may inhale quickly via the mouth (up to 1 second for example) followed by a breath-hold before exhaling through the nose or an external positive pressure may be applied to deliver the aerosol and then the patient exhales through the nose. In exemplary embodiments, slow inhalation may be up to 5 seconds while quick inhalation may be up to 1 second. The breath hold may be for up to 2 to 10 seconds. Other exemplary operating parameters are disclosed elsewhere in this specification. This method allows introduction of the particles or droplets into the respiratory tract with the desired momentum to facilitate deposition in the target region, and while preferably minimizing deposition in the mouth, peripheral lungs and anterior portion of the nasal cavity. Controlling momentum allows the excipient and/or therapeutic agent adequate residence time to be exposed to the natural relative humidity in the respiratory tract and therefore allows absorption of moisture by the excipient or therapeutic agent which causes the particles or droplets to growth to a desired size, thereby facilitating their deposition in the target region. This principle has been reported in the scientific and patent literature, and is referred to as excipient enhanced growth (EEG) and relies on the natural humidity of the environment in which the particles or droplets reside. Additionally, controlling the momentum ensures that the particles or droplets have a velocity that also facilitates deposition in the target tissue. If velocity is too high, the particles or droplets will not be able to follow the tortuous path of the respiratory tract. If velocity is too low, the particles or droplets may be deposited in unwanted regions of the respiratory tract.

In other embodiments, the patient may inhale through the first portion of the respiratory tract which may be the nose (also referred to as nasal inhalation), at the same flow rates, with the same breath holds as described previously and elsewhere in this specification, followed by exhalation through the nose.

Initial particle or droplet size is preferably in the range from about 1 μm to about 10 μm, and more preferably in the range from about 1 μm to about 8 μm. Thus the particles may initially be from 1 μm to 10 μm; 1 μm to 9 μm; 1 μm to 8 μm; 1 μm to 7 μm; 1 μm to 6 μm; 1 μm to 5 μm; 1 μm to 4 μm; 1 μm to 3 μm; or 1 μm to 2 μm. In other embodiments, the particles or droplets may have an initial size less than 1 μm, and in still other embodiments the initial size may be greater than 10 μm.

The therapeutic agent itself may be hygroscopic and therefore may grow upon absorption of moisture due to the natural relative humidity in the patient's respiratory tract, or the therapeutic agent may not be hygroscopic. The therapeutic agent may have an initial particle size in the range of 10 nm to 8 μm which is formulated within a combination particle or droplet containing one or more hygroscopic excipient or other excipients that are recognized by those skilled in the art as necessary to form stable droplets or particles such as surfactants, dispersion enhancers, bulking agents, lubricants. Use of a hygroscopic excipient therefore may help the particles or droplets grow. Growth of the particles or droplets is preferably in the range from about 2 to about 20 times the initial size. Thus growth, may be 2; 3; 4; 5; 6; 7; 8; 9; 10; 11; 12; 13; 14; 15; 16; 17; 18; 19; or 20 times the initial size. In other embodiments, growth may be greater than 20 times the initial size, and in still other embodiments growth may be less than 2 times initial size.

Particle or droplet sizes are generally referred to by their mass median aerodynamic diameter (MMAD) which is defined as the particle size at the 50^{th} percentile on a cumulative percentage mass undersize distribution using linear interpolation. This technical of characterizing particle size is well reported in the scientific and patent literature.

Any formulation of therapeutic agent and excipient may be used and customized in order control the growth ratio. For example, the ratio of excipient to therapeutic agent (w/w) may range from 0%:100% to 99%:1%, and everything in between. Thus this ratio may include 0%:100%; 5%:95%; 10%:90%; 15%:85%; 20%:80%; 25%:75: 30%:70%; 35%:65%; 40%:60%; 45%:55%: 50%:50%; 55%:45%; 60%:40%; 65%:35%; 70%:30%; 75%:25%; 80%:20%; 85%:15%; 90%:10%; 95%:5%; 99%:1%; and any ratios in between those.

### Exemplary Therapeutic Agents

Any number of therapeutic agents may be used with the devices and methods disclosed herein. Exemplary therapeutic agents include, but are not limited to those described below.

Substances (e.g. drugs, therapeutic agents, active agents, etc.) that may be formulated with a hygroscopic excipient as described herein or delivered as described herein include but are not limited to various agents, drugs, compounds, and compositions of matter or mixtures thereof that provide some beneficial pharmacologic effect. The particles of the invention broadly encompass substances including “small molecule” drugs, peptides, proteins, genes, vaccines, vitamins, nutrients, aroma-therapy substances, and other-beneficial agents. As used herein, the terms further include any physiologically or pharmacologically active substance that produces a localized or systemic effect in a patient, i.e. the agent may be active in the target tissue, or may be delivered to the target tissue as a gateway to systemic activity.

In some embodiments, the site of action of the substance that is delivered may be the nasal cavity, and more preferably is a portion of the nasal cavity posterior to the vestibule. Other portions of the respiratory tract may also be used as the target. Examples of such agents include but are not limited to agents for anesthesia; treatments for asthma or other respiratory conditions; anti-viral, anti-bacterial or anti-fungal agents; anti-cancer agents; α-1 antitrypsin and other antiproteases (for congenital deficiencies), rhDNAse (for cystic fibrosis), and cyclosporine (for lung transplantation), vaccines, proteins and peptides, etc. Other examples include bronchodilators including albuterol, terbutaline, isoprenaline and levalbuterol, and racemic epinephrine and salts thereof; anti-cholinergics including atropine, ipratropium bromide, tiatropium and salts thereof; expectorants including dornase alpha (pulmozyme) (used in the management of cystic fibrosis; corticosteroids such as budesonide, mometasone and its salts, triamcinolone and its salts, fluticasone and its salts; prophylactic anti-asthmatics such as sodium cromoglycate and nedocromil sodium; anti-infectives such as the antibiotic gentamicin and the anti-protozoan pentamidine (used in the treatment of Pneumocystis carinii pneumonia), and the antiviral agent ribavirin, used to treat respiratory syncytial virus e.g. in young children and infants.

However, this need not be the case. Some agents delivered via the nasal cavity into systemic circulation will be distributed systemically via the circulatory system. Examples of such agents include but are not limited to, for example, calcitonin (for osteoporosis), human growth hormone (HGH, for pediatric growth deficiency), various hormones such as parathyroid hormone (PTH, for hyperparathyroidism), insulin and other protein or peptide agents, nucleic acid molecules, and anti-pain or anti-inflammation agents. Such agents may require chronic administration. The ability of the invention to deliver these often expensive agents at higher delivery efficiencies to the nasal cavity where they are systemically absorbed is a significant advantage over conventional aerosol drug delivery methods including metered dose inhalers, dry powder inhalers and nebulizers.

Anti-infective agents may be required to treat localized infections within the airways. Targeting to specific regions within the nasal cavity and delivering drug aerosols with high deposition efficiencies is possible with this invention. Once a target region has been identified (through clinical examination), an aerosol would be produced that would have a final particle size suitable for deposition in that region. In this example, an initial aerosol would be formulated with appropriate hygroscopic excipients and inhaled. By controlling the amount of hygroscopic excipients present in the aerosol formulation, it is possible to control the final particle size of the aerosol and therefore ultimately its site of deposition within the nasal cavity.

Examples of anti-infective agents, whose class or therapeutic category is herein understood as comprising compounds which are effective against bacterial, fungal, and viral infections, i.e. encompassing the classes of antimicrobials, antibiotics, antifungals, antiseptics, and antivirals, are penicillins, including benzylpenicillins (penicillin-G-sodium, clemizone penicillin, benzathine penicillin G), phenoxypenicillins (penicillin V, propicillin), aminobenzylpenicillins (ampicillin, amoxycillin, bacampicillin), acylaminopenicillins (aziocillin, mezlocillin, piperacillin, apalcillin), carboxypcnicillins (carbenicillin, ticarcillin, temocillin), isoxazolyl penicillins (oxacillin, cloxacillin, dicloxacillin, flucloxacillin), and amidine penicillins (mecillinam); cephalosporins, including cefazolins (cefazolin, cefazedone); cefuroximes (cerufoxim, cefamdole, cefotiam), cefoxitins (cefoxitin, cefotetan, latamoxef, flomoxef), cefotaximes (cefotaxime, ceftriaxone, ceftizoxime, cefinenoxime), ceftazidimes (ceftazidime, cefpirome, cefepime), cefalexins (cefalexin, cefaclor, cefadroxil, cefradine, loracarbef, cefprozil), and cefiximes (cefixime, cefpodoxim proxetile, cefuroxime axetil, cefetamet pivoxil, cefotiam hexetil), loracarbef, cefepim, clavulanic acid/amoxicillin, Ceftobiprole; synergists, including beta-lactamase inhibitors, such as clavulanic acid, sulbactam, and tazobactam; carbapenems, including imipenem, cilastin, meropenem, doripenem, tebipenem, ertapenem, ritipenam, and biapenem; monobactams, including aztreonam; aminoglycosides, such as apramycin, gentamicin, amikacin, isepamicin, arbekacin, tobramycin, netilmicin, spectinomycin, streptomycin, capreomycin, neomycin, paromoycin, and kanamycin; macrolides, including erythromycin, clarythromycin, roxithromycin, azithromycin, dithromycin,josamycin, spiramycin and telithromycin; gyrase inhibitors or fluoroquinolones, including ciprofloxacin, gatifloxacin, norfloxacin, ofloxacin, levofloxacin, perfloxacin, lomefloxacin, fleroxacin, garenoxacin, clinafloxacin, silafloxacin, prulifloxacin, olamufloxacin, caderofloxacin, gemifloxacin, balofloxacin, trovafioxacin, and moxifloxacin; tetracyclins, including tetracyclin, oxytetracyclin, rolitetracyclin, minocyclin, doxycycline, tigecycline and aminocycline; glycopeptides, including vancomycin, teicoplanin, ristocetin, avoparcin, oritavancin, ramoplanin, and peptide 4; polypeptides, including plectasin, dalbavancin, daptomycin, oritavancin, ramoplanin, dalbavancin, telavancin, bacitracin, tyrothricin, neomycin, kanamycin, mupirocin, paromomycin, polymyxin B and colistin; sulfonamides, including sulfadiazine, sulfamethoxazole, sulfalene, co-trimoxazole, co-trimetrol, co-trimoxazine, and co-tetraxazine; azoles, including clotrimazole, oxiconazole, miconazole, ketoconazole, itraconazole, fluconazole, metronidazole, tinidazole, bifonazol, ravuconazol, posaconazol, voriconazole, and omidazole and other antifungals including flucytosin, griseofluvin, tonoftal, naftifin, terbinafin, amorolfin, ciclopiroxolamin, echinocandins, such as micafungin, caspofungin, anidulafungin; nitrofurans, including nitrofurantoin and nitrofuranzone; polyenes, including amphotericin B, natamycin, nystatin, flucocytosine; other antibiotics, including tithromycin, lincomycin, clindamycin, oxazolindiones (linzezolids), ranbezolid, streptogramine A+B, pristinamycin aA+B, Virginiamycin A+B, dalfopristin/qiunupristin (Synercid), chloramphenicol, ethambutol, pyrazinamid, terizidon, dapson, prothionamid, fosfomycin, fucidinic acid, rifampicin, isoniazid, cycloserine, terizidone, ansamycin, lysostaphin, iclaprim, mirocin B17, clerocidin, filgrastim, and pentamidine; antivirals, including aciclovir, ganciclovir, birivudin, valaciclovir, zidovudine, didanosin, thiacytidin, stavudin, lamivudin, zalcitabin, ribavirin, nevirapirin, delaviridin, trifluridin, ritonavir, saquinavir, indinavir, foscarnet, amantadin, podophyllotoxin, vidarabine, tromantadine, and proteinase inhibitors; plant extracts or ingredients, such as plant extracts from chamomile, hamamelis, echinacea, calendula, papain, pelargonium, essential oils, myrtol, pinen, limonen, cineole, thymol, mentol, alpha-hederin, bisabolol, lycopodin, vitapherole; wound healing compounds including dexpantenol, allantoin, vitamins, hyaluronic acid, alpha-antitrypsin, anorganic and organic zinc salts/compounds, interferones (alpha, beta, gamma), tumor necrosis factors, cytokines, interleukins.

In a similar way to that described for targeting antibiotics, it may also be desirable to target anti-cancer compounds or chemotherapy agents to tumors. It is envisaged that by formulating the agent with an appropriate hygroscopic growth excipient, it will be possible to target regions where it has been identified that the tumor is growing. Examples of suitable compounds are immunmodulators including methotrexat, azathioprine, cyclosporine, tacrolimus, sirolimus, rapamycin, mofetil, cytotatics and metastasis inhibitors, alkylants, such as nimustine, melphanlane, carmustine, lomustine, cyclophosphosphamide, ifosfamide, trofosfamide, chlorambucil, busulfane, treosulfane, prednimustine, thiotepa; antimetabolites, e.g. cytarabine, fluorouracil, methotrexate, mercaptopurine, tioguanine; alkaloids, such as vinblastine, vincristine, vindesine; antibiotics, such as alcarubicine, bleomycine, dactinomycine, daunorubicine, doxorubicine, epirubicine, idarubicine, mitomycine, plicamycine; complexes of secondary group elements (e.g. Ti, Zr, V, Nb, Ta, Mo, W, Pt) such as carboplatinum, cis-platinum and metallocene compounds such as titanocendichloride; amsacrine, dacarbazine, estramustine, etoposide, beraprost, hydroxycarbamide, mitoxanthrone, procarbazine, temiposide; paclitaxel, iressa, zactima, poly-ADP-ribose-polymerase (PRAP) enzyme inhibitors, banoxantrone, gemcitabine, pemetrexed, bevacizumab, ranibizumab may be added.

Additional active agents may be selected from, for example, hypnotics and sedatives, tranquilizers, anticonvulsants, muscle relaxants, antiparkinson agents (dopamine antagnonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents (particularly those that target lung cancer), antithrombotic agents, antihypertensives, cardiovascular drugs, antiarrhythmics, antioxicants, hormonal agents including contraceptives, sympathomimetics, diuretics, lipid regulating agents, antiandrugenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically. The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of other active agents suitable for use in this invention include but are not limited to one or more of calcitonin, amphotericin B, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha I, IIb/IIIa inhibitor, alpha-1 antitrypsin, phosphodiesterase (PDE) compounds, VLA-4 inhibitors, bisphosphonates, respiratory syncytial virus antibody, cystic fibrosis transmembrane regulator (CFTR) gene, deoxyreibonuclease (Dnase), bactericidal/permeability increasing protein (BPI), anti-CMV antibody, and 13-cis retinoic acid, and where applicable, analogues, agonists, antagonists, inhibitors, and pharmaceutically acceptable salt forms of the above. In reference to peptides and proteins, the invention may encompass synthetic, native, glycosylated, unglycosylated, pegylated forms, and biologically active fragments and analogs thereof. Active agents for use in the invention further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense and inhibitory RNA. Further, an active agent may comprise live attenuated or killed viruses suitable for use as vaccines. Other useful drugs include those listed within the Physician's Desk Reference (most recent edition).

An active agent for delivery or formulation as described herein may be an inorganic or an organic compound, including, without limitation, drugs which act on: the lung or other portions of the respiratory system such as nasal tissue, the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Frequently, the active agent acts in or on the nasal cavity.

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 99% by weight active agent, typically from about 2% to about 95% by weight active agent, and more typically from about 5% to 85% by weight active agent, and will also depend upon the relative amounts of hygroscopic excipient or other necessary excipient contained in the composition. The compositions of the invention are particularly useful for active agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one active agent may be incorporated into the formulations described herein and that the use of the term “agent” in no way excludes the use of two or more such agents.

In addition to one or more active agents and hygroscopic excipient(s), the aerosol particles/droplets may optionally include one or more pharmaceutical excipients (which differ from the hygroscopic excipients) that are suitable for nasal administration. These excipients, if present, are generally present in the composition in amounts ranging from about 0.01% to about 95% percent by weight, preferably from about 0.5 to about 80%, and more preferably from about 1 to about 60% by weight. Preferably, such excipients serve to further improve the features of the active agent composition, for example by improving the handling characteristics of powders, such as flowability and consistency, and/or facilitating manufacturing and filling of unit dosage forms. One or more excipients may also be provided to serve as bulking agents when it is desired to reduce the concentration of active agent in the formulation. One or more excipients may also be provided to serve as surfactants or solubilizing agents. Pharmaceutical excipients and additives useful in the present pharmaceutical formulation include but are not limited to amino acids, peptides, proteins, non-biological polymers, biological polymers, carbohydrates, such as sugars, dcrivatized sugars such as alditols, aldonic acids, esterified sugars, and sugar polymers, which may be present singly or in combination. The pharmaceutical formulation may also include a buffer or a pH adjusting agent, typically a salt prepared from an organic acid or base. Representative buffers include organic acid salts of citric acid, ascorbic acid, gluconic acid, carbonic acid, tartaric acid, succinic acid, acetic acid, or phthalic acid, Tris, tromethamine hydrochloride, or phosphate buffers. The pharmaceutical formulation may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-β-cyclodextrin and sulfobutylether-βcyclodextrin), polyethylene glycols, and pectin. The particles may further include inorganic salts, antimicrobial agents (for example benzalkonium chloride), antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations).

Drug substances that are particularly suitable for delivery using a hygroscopic excipient are generally particularly hydrophobic and/or that have a very low intrinsic capability to take on water. Such substances include but are not limited to corticosteroids, e.g. budesonide, fluticasone, triamcinolone and salts thereof; as well as certain benzodiazepines e.g. lorazepam, oxazepam, and temazepam.

The delivery devices and formulations of the invention are generally suitable for treating animals, preferably mammals. The mammal may be a human, but this is not always the case; veterinary applications and applications where animals are used to assess aerosol exposures to drugs and pollutants are also encompassed by the invention.

### Excipients

In addition to the excipients described above, any number of other excipients may be used with the therapeutic agents described above and in conjunction with the devices and methods disclosed herein. Exemplary excipients include those previously described above, as well as other salts, sugars, and acids. Additionally, any combination of therapeutic agents and excipient or excipients is possible. Exemplary excipients include, but are not limited to those described below.

Hygroscopic agents that may be used in the practice of the invention include but are not limited to: salts such as NaCl, KCl, zinc chloride, calcium chloride, magnesium chloride, potassium carbonate, potassium phosphate, carnallite, ferric ammonium citrate, magnesium sulfate, sodium sulfite, calcium oxide, ammonium sulfate; sugars such as sorbital, mannitol, glucose, maltose, galactose, fructose, sucrose; glycols such as polyethylene glycols (varying molecular weights), propylene glycol, glycerol; organic acids such as citric acid, sulfuric acid, malonic acid, adipic acid; lactams such as 2-pyrrolidone, polyvinylpolyprrolidone (PVP); other substances include potassium hydroxide, sodium hydroxide, gelatin, hydroxypropyl methylcellulose, pullalan, starch, polyvinyl alcohol, and sodium cromoglycate.

The amount of hygroscopic excipient that is formulated with the therapeutic substance, either as a dry powder particle or in a formulated drug solution from which aerosolized droplets are generated generally ranges from about 1% by weight to about 99% by weight, typically from about 2% to about 95% by weight, and more typically from about 5% to 85% by weight (i.e. % of the total particle weight). The amount varies depending on several factors. The amount varies, e.g. according to the type of therapeutic agent(s) (more than one therapeutic substance may be present in a particle/droplet) and/or other substances (and other substances such as buffering substances, bulking agents, wetting agents, etc.), that are present in the particle/droplet, as well as the particular hygroscopic excipient that is used. In addition, the ratio of drug and hygroscopic excipient present in the initial particle or droplet is determined by the rate and extent of aerosol particle size growth that is required to target deposition sites for the aerosol particles within the airways.

As previously mentioned, the aerosol generator may be any generator, such as a metered dose inhaler, dry powder inhaler, liquid spray, jet nebulizer, capillary aerosol generator, condensational aerosol generator, or an ultrasonic nebulizer. While the aerosol is delivered preferably in a bolus or intermittent boluses, a continuous aerosol generator may also be used, or the continuous aerosol generator may be actuated intermittently to provide a desired cycle of aerosol.

### Exemplary Protocols

Computational fluid dynamic models were used to evaluate several exemplary protocols of oral inhalation of an aerosol containing particles or droplets followed by nasal exhalation. These models predict the deposition of the particles or droplets in along various portions of the respiratory tract.

### Protocol 1

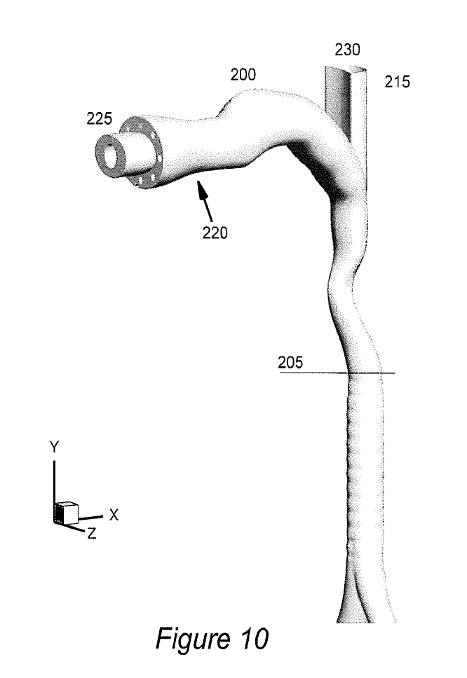

In Protocol 1, a Respimat inhaler is used as the aerosol generator. The patient takes a breath that is a normal breath or approximately 90% of a normal tidal volume. The inhaler spacer is then positioned in the patient's mouth and the Respimat is fired for approximately 1.5 seconds. As examples of this protocol, the initial particle size of the aerosol was varied using particle size of 3 μm and 5.3 μm MMAD, respectively. Next, approximately 50 cc of air is injected or inhaled through the air inlet for controlled volume from a syringe or inhaled from a gas bag over approximately 3 seconds to produce a flow rate of about 1 liter per minute. The cumulative time is now about 4.5 seconds. The patient then exhales through the nose at a flow rate of about 60 liters per minute with the inhaler remaining in place to prevent oral exhalation. The cumulative time is now about 6 seconds. This allows residence time for the particles or droplets to grow and deposit in desired target regions of the nasal cavity or other regions of the respiratory tract. In this protocol, the injected 50 cc of air is inhaled into the respiratory tract but generally does not enter the lungs. FIG. 10 schematically illustrates the experimental setup of a mouth-throat MT model 200 consisting of elliptical mouth throat geometry ending at line 205, characteristic tracheobronchial TB geometry 210 through the main bifurcation, and a characteristic lower nasopharynx NP 215 region, below the outlet to the nasal cavity 230, together with the spacer 220 and inlet 225 of the Respimat. The inlet is for injection of a controlled volume of air from a chamber. The air may be injected from a chamber or other structure having a defined volume. Additional details on this feature will be described later in this application. The inhaled volume is low to keep the aerosol in the upper trachea-bronchial (TB) airways, also to foster excipient enhanced growth, and also to minimize depositional loss. Subsequent nasal exhalation then fosters deposition in the nasal passage. Table 1 below summarizes this protocol as well as other exemplary protocols.

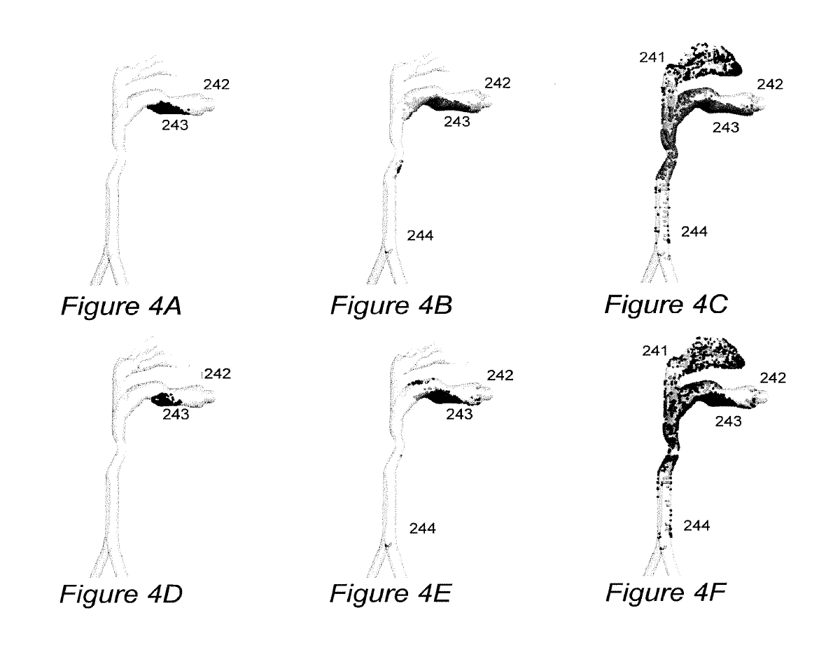

FIGS. 4A-4C illustrate the deposition results predicted using computational fluid dynamic modeling under Protocol 1 and with 5.3 μm size particles. FIG. 4A shows that there is approximately 0.84% deposition fraction in the spacer (242) after Step 2 and approximately 1.80% deposition fraction in the mouth-throat region (243). The cumulative time after Step 2 is about 1.5 seconds. FIG. 4B illustrates increased deposition fraction in the spacer (242) of about 4.00%, 7.41% deposition fraction in the mouth-throat region (243), and about 0.20% deposition fraction in the trachea-bronchial region (244), all after Step 3 which is with a cumulative time of about 4.5 seconds. Finally, FIG. 4C illustrates the modeling results at the end of Step 4, or after a cumulative time of about 6 seconds. The deposition fraction in the nasal cavity (241) is about 18.56%, while the deposition fraction in the spacer (242) is about 4.58%. The deposition fraction in the mouth-throat region (243) is about 22.97% and the deposition fraction in the trachea-bronchial region (244) is about 1.1%. Therefore, particles or droplets are clearly depositing in the nasal region as desired.

FIGS. 4D-4F illustrate the deposition results predicted using computational fluid dynamic modeling under Protocol 1 with 3 μm size particles. FIG. 4D shows that there is approximately 0.14% deposition fraction in the spacer (242) after Step 2 and approximately 0.68% deposition fraction in the mouth-throat region (243). The cumulative time after Step 2 is about 1.5 seconds. FIG. 4E illustrates increased deposition fraction in the spacer (242) of about 1.24%, 2.95% deposition fraction in the mouth-throat region (243), and about 0.17% deposition fraction in the trachea-bronchial region (244), all after Step 3 which is with a cumulative time of about 4.5 seconds. Finally, FIG. 4F illustrates the modeling results at the end of Step 4, or after a cumulative time of about 6 seconds. The deposition fraction in the nasal cavity (241) is about 12.33%, while the deposition fraction in the spacer (242) is about 1.57%. The deposition fraction in the mouth-throat region (243) is about 12.36% and the deposition fraction in the trachea-bronchial region (244) is about 0.78%. Therefore, particles or droplets appear to have a higher deposition fraction in the nasal area for 5.3 μm size particles versus the 3 μm particles.

### Protocol 2.1

In Protocol 2.1, a Respimat inhaler is used as the aerosol generator. The patient takes a breath that is a normal breath or approximately 90% of a normal tidal volume. The inhaler spacer is then positioned in the patient's mouth and the Respimat is fired for approximately 1.5 seconds. As examples of this protocol, the initial particle size of the aerosol was varied using particle size of 3 μm and 5.3 μm MMAD, respectively. Next, approximately 50 cc of air is injected or inhaled through the air inlet for controlled volume from a syringe or inhaled from a gas bag over approximately 1 second to produce a flow rate of about 3 liters per minute. The cumulative time is now about 2.5 seconds. The patient then exhales through the nose at a flow rate of about 60 liters per minute with the inhaler remaining in place to prevent oral exhalation. The exhaled volume is about 3 liters, and the cumulative time is now about 5.5 seconds. This allows residence time for the particles or droplets to grow and deposit in desired target regions of the nasal cavity or other regions of the respiratory tract. In this protocol, the injected 50 cc of air is inhaled into the respiratory tract but generally docs not enter the lungs. FIG. 10 schematically illustrates the experimental setup of a mouth-throat MT model 200 consisting of elliptical mouth throat geometry ending at line 205, characteristic tracheobronchial TB geometry 210 through the main bifurcation, and a characteristic lower nasopharynx NP 215 region together with the spacer 220 and inlet 225 of the Respimat. The air may be injected from a chamber or other structure having a defined volume. Additional details on this feature will be described later in this application. The inhaled volume is low to keep the aerosol in the upper trachea-bronchial (TB) airways, also to foster excipient enhanced growth, and also to minimize depositional loss. Subsequent nasal exhalation then fosters deposition in the nasal passage. Table 1 below summarizes this protocol as well as other exemplary protocols.

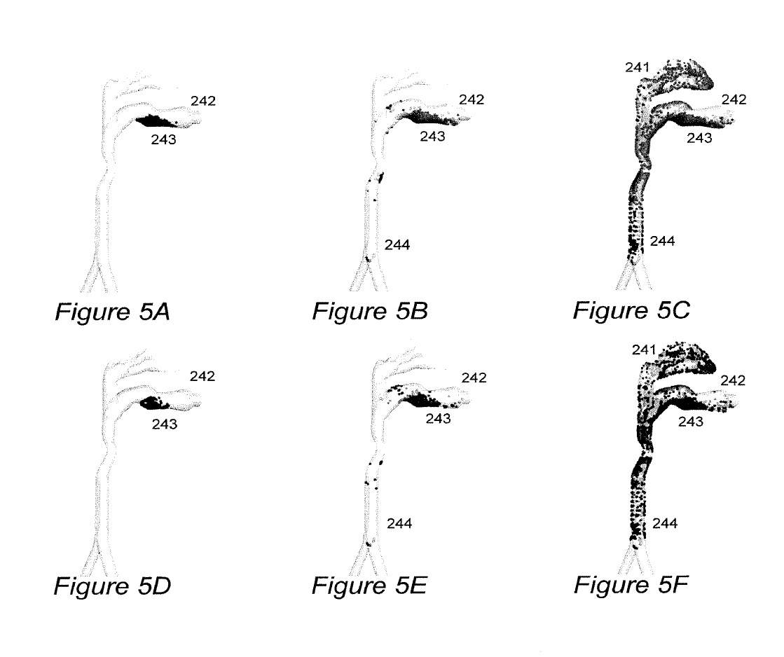

FIGS. 5A-5C illustrate the deposition results predicted using computational fluid dynamic modeling under Protocol 2.1 and with 5.3 μm size particles. FIG. 5A shows that there is approximately 0.84% deposition fraction in the spacer (242) after Step 2 and approximately 1.80% deposition fraction in the mouth-throat region (243); The cumulative time after Step 2 is about 1.5 seconds. FIG. 5B illustrates increased deposition fraction in the spacer (242) of about 2.33%, 4.70% deposition fraction in the mouth-throat region (243), and about 0.68% deposition fraction in the trachea-bronchial region (244), all after Step 3 which is with a cumulative time of about 2.5 seconds. Finally, FIG. 5C illustrates the modeling results at the end of Step 4, or after a cumulative time of about 5.5 seconds. The deposition fraction in the nasal cavity (241) is about 19.69%, while the deposition fraction in the spacer (242) is about 3.10%. The deposition fraction in the mouth-throat region (243) is about 20.40% and the deposition fraction in the trachea-bronchial region (244) is about 3.94%. Therefore, particles or droplets are clearly depositing in the nasal region as desired.

FIGS. 5D-5F illustrate the deposition results predicted using computational fluid dynamic modeling under Protocol 2.1 with 3 μm size particles. FIG. 5D shows that there is approximately 0.14% deposition fraction in the spacer (242) after Step 2 and approximately 0.68% deposition fraction in the mouth-throat region (243). The cumulative time after Step 2 is about 1.5 seconds. FIG. 5E illustrates increased deposition fraction in the spacer (242) of about 0.53%, 1.86% deposition fraction in the mouth-throat region (243), and about 0.66% deposition fraction in the trachea-bronchial region (244), all after Step 3 which is with a cumulative time of about 2.5 seconds. Finally, FIG. 5F illustrates the modeling results at the end of Step 4, or after a cumulative time of about 5.5 seconds. The deposition fraction in the nasal cavity (241) is about 12.23%, while the deposition fraction in the spacer (242) is about 1.03%. The deposition fraction in the mouth-throat region (243) is about 13.92% and the deposition fraction in the trachea-bronchial region (244) is about 3.62%. Therefore, particles or droplets appear to have a higher deposition fraction in the nasal area for 5.3 μm size particles versus the 3 μm particles.

### Protocol 2.2

In Protocol 2.2, a Respimat inhaler is used as the aerosol generator. The patient takes a breath that is a normal breath or approximately 90% of a normal tidal volume. The inhaler spacer is then positioned in the patient's mouth and the Respimat is fired for approximately 1.5 seconds. As examples of this protocol, the initial particle size of the aerosol was varied using particle size of 3 μm and 5.3 μm MMAD, respectively. Next, approximately 75 cc of air is injected or inhaled through the air inlet for controlled volume from a syringe or inhaled from a gas bag over approximately 1 second to produce a flow rate of about 4.5 liters per minute. The cumulative time is now about 2.5 seconds. The patient then exhales through the nose at a flow rate of about 60 liters per minute with the inhaler remaining in place to prevent oral exhalation. The exhaled volume is about 3 liters, and the cumulative time is now about 5.5 seconds. This allows residence time for the particles or droplets to grow and deposit in desired target regions of the nasal cavity or other regions of the respiratory tract. In this protocol, the injected 75 cc of air is inhaled into the respiratory tract but generally does not enter the lungs. FIG. 10 schematically illustrates the experimental setup of a mouth-throat MT model 200 consisting of elliptical mouth throat geometry ending at line 205, characteristic tracheobronchial TB geometry 210 through the main bifurcation, and a characteristic lower nasopharynx NP 215 region together with the spacer 220 and inlet 225 ofthe Respimat. The air may be injected from a chamber or other structure having a defined volume. Additional details on this feature will be described later in this application. The inhaled volume is low to keep the aerosol in the upper trachea-bronchial (TB) airways, also to foster excipient enhanced growth, and also to minimize depositional loss. Subsequent nasal exhalation then fosters deposition in the nasal passage. Table 1 below summarizes this protocol as well as other exemplary protocols.

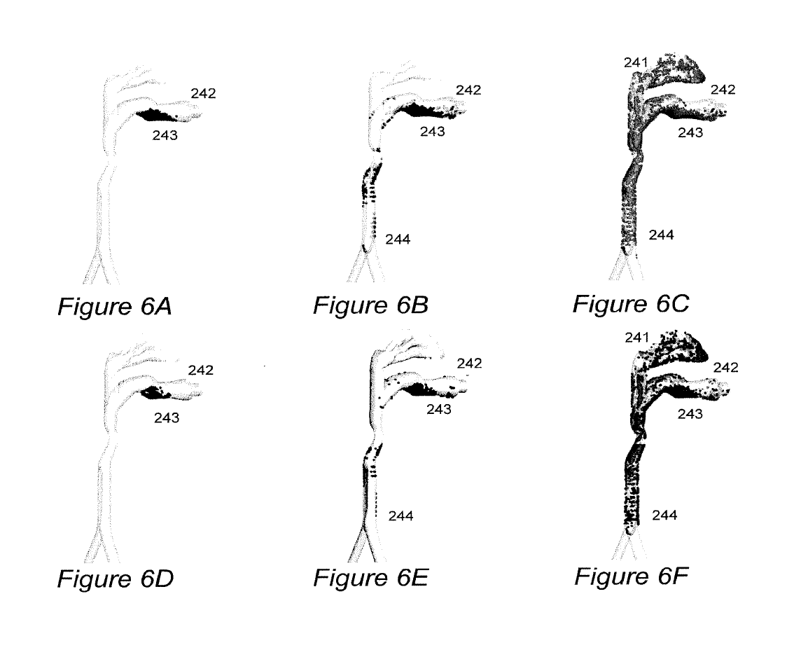

FIGS. 6A-6C illustrate the deposition results predicted using computational fluid dynamic modeling under Protocol 2.2 and with 5.3 μm size particles. FIG. 6A shows that there is approximately 0.84% deposition fraction in the spacer (242) after Step 2 and approximately 1.80% deposition fraction in the mouth-throat region (243). The cumulative time after Step 2 is about 1.5 seconds. FIG. 6B illustrates increased deposition fraction in the spacer (242) of about 2.17%, 5.18% deposition fraction in the mouth-throat region (243), and about 2.22% deposition fraction in the trachea-bronchial region (244), all after Step 3 which is with a cumulative time of about 2.5 seconds. Finally, FIG. 6C illustrates the modeling results at the end of Step 4, or after a cumulative time of about 5.5 seconds. The deposition fraction in the nasal cavity (241) is about 18.74%, while the deposition fraction in the spacer (242) is about 2.68%. The deposition fraction in the mouth-throat region (243) is about 20.78% and the deposition fraction in the trachea-bronchial region (244) is about 6.05%. Therefore, particles or droplets are clearly depositing in the nasal region as desired.

FIGS. 6D-6F illustrate the deposition results predicted using computational fluid dynamic modeling under Protocol 2.2 with 3 μm size particles. FIG. 6D shows that there is approximately 0.14% deposition fraction in the spacer (242) after Step 2 and approximately 0.68% deposition fraction in the mouth-throat region (243). The cumulative time after Step 2 is about 1.5 seconds. FIG. 6E illustrates increased deposition fraction in the spacer (242) of about 0.57%, 2.33% deposition fraction in the mouth-throat region (243), and about 1.66% deposition fraction in the trachea-bronchial region (244), all after Step 3 which is with a cumulative time of about 2.5 seconds. Finally, FIG. 6F illustrates the modeling results at the end of Step 4, or after a cumulative time of about 5.5 seconds. The deposition fraction in the nasal cavity (241) is about 11.24%, while the deposition fraction in the spacer (242) is about 0.93%. The deposition fraction in the mouth-throat region (243) is about 14.35% and the deposition fraction in the trachea-bronchial region (244) is about 4.88%. Therefore, particles or droplets appear to have a higher deposition fraction in the nasal area for 5.3 μm size particles versus the 3 μm particles.

### Protocol 2.3

In Protocol 2.3, a Respimat inhaler is used as the aerosol generator. The patient takes a breath that is a normal breath or approximately 90% of a normal tidal volume. The inhaler spacer is then positioned in the patient's mouth and the Respimat is fired for approximately 1.5 seconds. As examples of this protocol, the initial particle size of the aerosol was varied using particle size of 3 μm and 5.3 μm MMAD, respectively. Next, approximately 100 cc of air is injected or inhaled through the air inlet for controlled volume from a syringe or inhaled from a gas bag over approximately 1 second to produce a flow rate of about 6 liters per minute. The cumulative time is now about 2.5 seconds. The patient then exhales through the nose at a flow rate of about 60 liters per minute with the inhaler remaining in place to prevent oral exhalation. The exhaled volume is about 3 liters, and the cumulative time is now about 5.5 seconds. This allows residence time for the particles or droplets to grow and deposit in desired target regions of the nasal cavity or other regions of the respiratory tract. In this protocol, the injected 100 cc of air is inhaled into the respiratory tract but generally does not enter the lungs. FIG. 10 schematically illustrates the experimental setup of a mouth-throat MT model 200 consisting of elliptical mouth throat geometry ending at line 205, characteristic tracheobronchial TB geometry 210 through the main bifurcation, and a characteristic lower nasopharynx NP 215 region together with the spacer 220 and inlet 225 of the Respimat. The air may be injected from a chamber or other structure having a defined volume. Additional details on this feature will be described later in this application. The inhaled volume is low to keep the aerosol in the upper trachea-bronchial (TB) airways, also to foster excipient enhanced growth, and also to minimize depositional loss. Subsequent nasal exhalation then fosters deposition in the nasal passage. Table 1 below summarizes this protocol as well as other exemplary protocols.

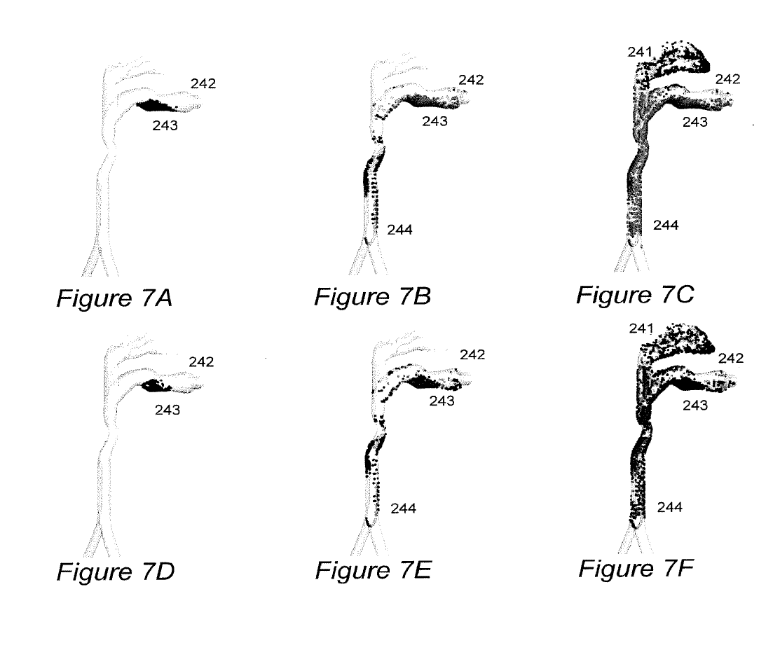

FIGS. 7A-7C illustrate the deposition results predicted using computational fluid dynamic modeling under Protocol 2.3 and with 5.3 μm size particles. FIG. 7A shows that there is approximately 0.84% deposition fraction in the spacer (242) after Step 2 and approximately 1.80% deposition fraction in the mouth-throat region (243). The cumulative time after Step 2 is about 1.5 seconds. FIG. 7B illustrates increased deposition fraction in the spacer (242) of about 2.11%, 5.65% deposition fraction in the mouth-throat region (243), and about 3.35% deposition fraction in the trachea-bronchial region (244), all after Step 3 which is with a cumulative time of about 2.5 seconds. Finally, FIG. 7C illustrates the modeling results at the end of Step 4, or after a cumulative time of about 5.5 seconds. The deposition fraction in the nasal cavity (241) is about 11.85%, while the deposition fraction in the spacer (242) is about 2.54%. The deposition fraction in the mouth-throat region (243) is about 13.85% and the deposition fraction in the trachea-bronchial region (244) is about 5.51%. Therefore, particles or droplets are clearly depositing in the nasal region as desired.

FIGS. 7D-7F illustrate the deposition results predicted using computational fluid dynamic modeling under Protocol 2.3 with 3 μm size particles. FIG. 7D shows that there is approximately 0.14% deposition fraction in the spacer (242) after Step 2 and approximately 0.68% deposition fraction in the mouth-throat region (243). The cumulative time after Step 2 is about 1.5 seconds. FIG. 7E illustrates increased deposition fraction in the spacer (242) of about 0.57%, 2.87% deposition fraction in the mouth-throat region (243), and about 2.45% deposition fraction in the trachea-bronchial region (244), all after Step 3 which is with a cumulative time of about 2.5 seconds. Finally, FIG. 7F illustrates the modeling results at the end of Step 4, or after a cumulative time of about 5.5 seconds. The deposition fraction in the nasal cavity (241) is about 7.33%, while the deposition fraction in the spacer (242) is about 1.02%. The deposition fraction in the mouth-throat region (243) is about 10.24% and the deposition fraction in the trachea-bronchial region (244) is about 4.44%. Therefore, particles or droplets appear to have a higher deposition fraction in the nasal area for 5.3 μm size particles versus the 3 μm particles.

TABLE 1
Delivery protocols for oral aerosol inhalation followed by nasal exhalation

| | Protocol 1 | Protocol 2 | | |
|---|---|---|---|---|
| | 1.1 | 2.1 | 2.2 | 2.3 |
| Step 1: | 90% inhalation prior | 90% inhalation prior | 90% inhalation prior | 90% inhalation prior |
| Preparations | to inhaler use (take a | to inhaler use (take a | to inhaler use (take a | to inhaler use (take a |
| | normal breath) | normal breath) | normal breath) | normal breath) |
| Step 2: Aerosol | Position inhaler in | Position inhaler in | Position inhaler in | Position inhaler in |
| generation | mouth | mouth | mouth | mouth |
| | Fire Respimat inhaler | Fire Respimat | Fire Respimat | Fire Respimat |
| | (1.5 s) | inhaler (1.5 s) | inhaler (1.5 s) | inhaler (1.5 s) |
| | MMAD = 3 or 5.3 μm | MMAD = 3 or 5.3 μm | MMAD = 3 or 5.3 μm | MMAD = 3 or 5.3 μm |
| | Count to 3 | Count to 3 | Count to 3 | Count to 3 |
| | Cumulative time: 1.5 s | Cumulative time: 1.5 s | Cumulative time: 1.5 s | Cumulative time: 1.5 s |
| Step 3: | Inject 50 cc of air | Inject 50 cc of air | Inject 75 cc of air | Inject 100 cc of air |
| Inhalation | from the syringe over | from the syringe | from the syringe | from the syringe |
| maneuver | 3 s (1 LPM) | over 1 s (3 LPM) | over 1 s (4.5 LPM) | over 1 s (6 LPM) |
| | Cumulative time: 4.5 s | Cumulative time: 2.5 s | Cumulative time: 2.5 s | Cumulative time: 2.5 s |
| Step 4: | Exhale through the | Exhale through | Exhale through | Exhale through |
| Exhalation | nose (~60 LPM) with | the nose (~60 | the nose (~60 | the nose (~60 |
| maneuver | inhaler in place to | LPM) with inhaler | LPM) with inhaler | LPM) with inhaler |
| | prevent oral exhalation | in place to prevent | in place to prevent | in place to prevent |
| | Cumulative time: 6.0 s | oral exhalation | oral exhalation | oral exhalation |
| | | (V = 3 L) | (V = 3 L) | (V = 3 L) |
| | | Cumulative time: | Cumulative time: | Cumulative time: |
| | | 5.5 s | 5.5 s | 5.5 s |

### Chamber

In various embodiments described above, the exemplary methods employed the use of an air chamber or a chamber and a spacer to control the flow rate and volume of air injected into the patient along with the aerosol.

In the exemplary protocols described above, an air spacer is placed between the aerosol generator and the patient's mouth. The spacer has a volume that minimizes deposition of the aerosol. In alternative embodiments, a bag or chamber having a volume of air or other gas is preferably placed behind the aerosol generator. This volume of air may be ambient air or it may be conditioned (heated or humidified) and is pushed or pulled through the aerosol generator when the patient inhales.

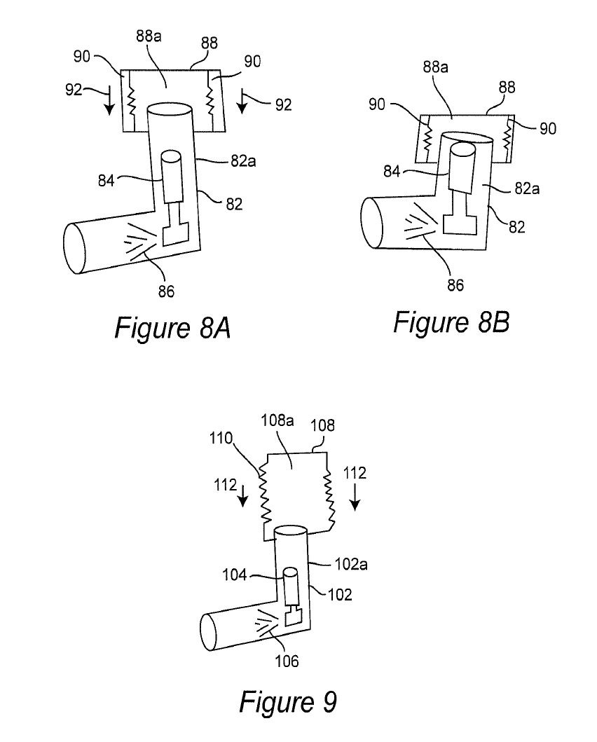

FIGS. 8A-8B illustrate actuation of an exemplary embodiment of a chamber. The inhaler 82 includes a generator 84 for producing an aerosol 86 and an outer sheath 82a for directing the aerosol to the patient's mouth. A housing 88 may be slidably or otherwise actuatably coupled to the inhaler 82 and a fixed volume of air or other gas 88a may be contained in the housing 88. The housing 88 may be spring loaded with springs 90 such that it is biased to return to an unactuated position. When the patient inhales, the housing actuates downward 92 and the fixed volume of air is pulled through the generator and delivered to the patient via outer sheath 82a to the patient's mouth. FIG. 8B illustrates the chamber after it has been actuated and the fixed volume of air has been delivered. The springs will return the housing to its unbiased position.

FIG. 9 illustrates another exemplary embodiment of an air spacer. The inhaler 102 includes a generator 104 for producing an aerosol 106 and an outer sheath 102a for directing the aerosol to the patient's mouth. A flexible chamber, such as a bag 108 is coupled to the inhaler 102. The flexible chamber may be a bag, or other member for containing a fixed volume of gas 108a. Corrugations 110 may be used to help the bag expand and collapse during downward actuation 112. Actuation of generator causes the fixed volume of air in the bag to be delivered through the generator to the patient's mouth via sheath 102a.

In the embodiments of FIGS. 8A-8B and FIG. 9, the air spacer is positioned behind the aerosol generator so that it does not change particle size distribution from the inhaler to the patient's mouth. However, one of skill in the art will appreciate that the air spacer could be disposed in front of the aerosol generator. Additional embodiments of the chamber are illustrated in FIG. 10, when positioned in the mouth of a model airway geometry.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of delivering a therapeutic agent to targeted regions of a nasal cavity, said method comprising:
providing a generator for generating an aerosol comprising particles or droplets containing a therapeutic agent and one or more excipients;
delivering the aerosol to a first portion of a respiratory tract in the patient, wherein particles or droplets have an initial diameter from about 1 μm to about 8 μm, and wherein an initial momentum of the particles or droplets minimizes deposition of the particles or droplets in the first portion of the respiratory tract;
exposing the particles or droplets to relative humidity in the respiratory tract by delivering the particles or droplets at a flow rate with an optional breath hold that defines the residence time of the particles or droplets in the respiratory tract;
increasing diameter of the particles or droplets due to hygroscopic growth caused by the relative humidity and residence time in the respiratory tract;
nasally exhaling a volume of the aerosol sufficient to deliver particles or droplets to the nasal cavity and minimize pulmonary delivery thereof; and
depositing the increased diameter particles or droplets in the nasal turbinates and/or the sinus cavities of the nasal cavity enhanced by the increased diameter of the particles or droplets.

2. The method of claim 1, wherein the particles or droplets further comprise a hygroscopic excipient.

3. The method of claim 2, wherein the hygroscopic excipient comprises a salt, a sugar, an acid, a buffer, a glycol, or a lactam.

4. The method of claim 2, wherein the hygroscopic excipient comprises one or more of the following materials: sodium chloride, sodium citrate, citric acid, potassium chloride, zinc chloride, calcium chloride, magnesium chloride, potassium carbonate, potassium phosphate, carnallite, ferric ammonium citrate, magnesium sulfate, sodium sulfite, calcium oxide, ammonium sulfate, sorbital, mannitol, glucose, maltose, galactose, fructose, sucrose, polyethylene glycol, propylene glycol, glycerol, sulfuric acid, malonic acid, adipic acid; lactams such as 2-pyrrolidone, polyvinylpolyprrolidone (PVP), potassium hydroxide, sodium hydroxide, gelatin, hydroxypropyl methylcellulose, pullalan, starch, polyvinyl alcohol, and sodium cromoglycate.

5. The method of claim 1, wherein the therapeutic agent does not promote hygroscopic growth of the particles or droplets.

6. The method of claim 1, wherein the initial momentum of the particles or droplets permits substantially unimpeded travel of the particles or droplets through the first portion of the respiratory tract.

7. The method of claim 1, wherein the increased diameter of the particles or droplets substantially enhances deposition of the particles or droplets in the nasal turbinates and/or sinus cavities.

8. The method of claim 1, wherein the therapeutic agent is selected from the group consisting of agents for the treatment of asthma, rhinitis, chronic sinusitis and other respiratory disorders, anesthesia agents, nucleic acid molecules, anti-pain agents, anti-inflammation agents, anti-depressants and other mood altering drugs, anti-viral agents, anti-bacterial agents, anti-fungal agents, anti-cancer agents, hormones, benzodiazepines and calcitonin.

9. The method of claim 1, wherein the relative humidity is a natural relative humidity in a portion of the respiratory tract.

10. The method of claim 1, wherein the ratio of the increased diameter to the initial diameter ranges from about 2 to about 20.

11. The method of claim 1, wherein the initial momentum is consistent with the inhalation of the 1-8 μm particles or droplets at a flow rate of less than about 30 liters per minute.

12. The method of claim 1, wherein the first portion of the respiratory tract is a mouth, an oropharyngeal region, a trachea, a pharynx, or a nasal cavity of the patient.

13. The method of claim 1, wherein the aerosol is contained in a fixed volume for delivery to the first portion of the respiratory tract.

14. The method of claim 1, wherein the aerosol is contained in a fixed volume for delivery to the first portion of a nasal cavity.

15. A device for delivering aerosolized particles or droplets to a region of a nasal passageway in a patient, said device comprising:
an aerosol generator;
an apparatus to control a volume of aerosol introduced to a respiratory tract from the aerosol generator;
a therapeutic agent and a hygroscopic excipient contained in the aerosol generator, wherein the aerosol generator generates an aerosol of particles or droplets containing the therapeutic agent and the hygroscopic excipient,
wherein the particles or droplets have an initial momentum while increasing in diameter to permit substantially unimpeded travel of the particles or the droplets through a first portion of the respiratory tract without significant deposition, and
wherein exposure of the particles or droplets to relative humidity in the respiratory tract increases diameter of the particles or droplets to a size that generally favors deposition in the nasal passageway, and
wherein the increased diameter of the particles or droplets results in deposition of the particles or droplets in the nasal turbinates and/or the sinus cavities of the nasal passageway.

16. The device of claim 15, wherein the apparatus that controls the volume of aerosol also controls the rate of aerosol delivery to the respiratory tract.

17. The device of claim 15, wherein the apparatus that controls the volume of aerosol also injects a volume of gas at positive pressure.

18. The device of claim 15, wherein the apparatus that controls the volume of aerosol limits air inhaled by the patient.

19. The device of claim 18, wherein the apparatus that controls the volume of aerosol comprises a bag pre-filled with the air inhaled by the patient.

20. The device of claim 18, wherein the air inhaled by the patient is limited to from about 25 cc to about 250 cc.

21. The device of claim 15, wherein the therapeutic agent is selected from the group consisting of agents for the treatment of asthma and other respiratory disorders, anesthesia agents, nucleic acid molecules, anti-pain agents, anti-inflammation agents, anti-depressants and other mood altering drugs, anti-viral agents, anti-bacterial agents, anti-fungal agents, anti-cancer agents, hormones, benzodiazepines and calcitonin.

22. The device of claim 15, wherein the therapeutic agent does not promote hygroscopic growth of the particles or droplets.

23. The device of claim 15, wherein the hygroscopic excipient comprises a salt, a sugar, an acid, a buffer, a glycol, or a lactam.

24. The device of claim 15, wherein the hygroscopic excipient comprises one or more of the following materials: sodium chloride, sodium citrate, citric acid, potassium chloride, zinc chloride, calcium chloride, magnesium chloride, potassium carbonate, potassium phosphate, carnallite, ferric ammonium citrate, magnesium sulfate, sodium sulfite, calcium oxide, ammonium sulfate, sorbital, mannitol, glucose, maltose, galactose, fructose, sucrose, polyethylene glycol, propylene glycol, glycerol, sulfuric acid, malonic acid, adipic acid; lactams such as 2-pyrrolidone, polyvinylpolyprrolidone (PVP), potassium hydroxide, sodium hydroxide, gelatin, hydroxypropyl methylcellulose, pullalan, starch, polyvinyl alcohol, and sodium cromoglycate.

25. The device of claim 15, wherein the ratio of the increased diameter to the initial diameter ranges from about 2 to about 20.

26. The device of claim 15, wherein the device delivers a fixed volume of the aerosol to the respiratory tract, the fixed volume ranging from about 25 cc to about 250 cc.

27. The device of claim 15, wherein the device controls flow rate of the aerosol delivered to the respiratory tract, the flow rate ranging from about 1 liter per minute to about 30 liters per minute, and wherein the flow rate minimizes pulmonary deposition.

28. The device of claim 15, wherein the particles or droplets are also deposited in a nasal ostium, a nasopharynx, an olfactory region, or in an area posterior relative to the vestibule.

29. The device of claim 15, wherein the aerosol generator is selected from the group consisting of a metered dose inhaler, a dry powder inhaler, a liquid spray device, a capillary aerosol generator, a condensational aerosol generator, a jet nebulizer, and an ultrasonic nebulizer.

30. The device of claim 29, wherein the aerosol generator provides a bolus of aerosol.

31. The device of claim 30, wherein the bolus of aerosol is an intermittent bolus of aerosol.

32. Use of a therapeutic agent and an excipient for treating diseases, wherein the therapeutic agent and the excipient are delivered from a generator to form particles or droplets delivered in an aerosol to targeted nasal tissue along a nasal passageway in a patient, the particle or droplets having an initial momentum to minimize deposition of the particles or droplets in a first region of a respiratory tract away from the targeted nasal tissue, and
wherein the particles or droplets increase in diameter when exposed to relative humidity in a portion of the nasal passageway, and
wherein the increased diameter of the particle or droplets enhances deposition of the particles in the targeted nasal tissue, and
wherein the targeted nasal tissue comprises the nasal turbinates and/or sinus cavities of the nasal passageway.

33. The use of the therapeutic agent and the excipient as in claim 32, wherein the therapeutic agent is selected from the group consisting of agents for the treatment of asthma and other respiratory disorders, anesthesia agents, nucleic acid molecules, anti-pain agents, anti-inflammation agents, anti-depressants and other mood altering drugs, anti-viral agents, anti-bacterial agents, anti-fungal agents, anti-cancer agents, hormones, benzodiazepines and calcitonin.

34. The use of the therapeutic agent and the excipient as in claim 32, wherein the ratio of the increased diameter to the initial diameter ranges from about 2 to about 20.

35. A method of providing one or more agents to a posterior region of a subject's nose, comprising the steps of:
generating an aerosol comprising a plurality of particles or droplets containing said one or more agents and optionally one or more excipients, said particles or droplets having a diameter ranging from 1 μm to 8 μm upon generation, and said particles or droplets having a property of hygroscopic growth when exposed to a humid environment;
delivering said aerosol to said subject's respiratory tract at a predetermined air flow rate, said delivering being performed for a period sufficient to at least partially fill one or more of said subject's nasal cavity, pharynx, larynx, and trachea, said particles or droplets experiencing hygroscopic growth due to exposure to relative humidity in one or more of said subject's nasal cavity, pharynx, larynx, and trachea; and
after said period, having said subject exhale through said subject's nose, said exhalation causing a majority of said particles or droplets to deposit in said posterior region of said subject's nose.

36. The method of claim 35, wherein the predetermined flow rate is front about 1 to 30 liters per minute.
